(19) United States
(12) Patent Application Publication
(10) Pub. No.: US 2004/0229795 A1
(43) Pub. Date: Nov. 18, 2004

(54) Use of antithrombin III for the prophylaxis and therapy of diseases

(75) Inventors: Juergen Roemisch, Marburg (DE); Harald Stauss, Dautphetal (DE); Christian Josef Wiedermann, Innsbruck (AT)

Correspondence Address: FINNEGAN, HENDERSON, FARABOW, GARRETT & DUNNER, LLP, 1300 I STREET, NW, WASHINGTON, DC, 20005, US

(73) Assignee: Aventis Behring GmbH

(21) Appl. No.: 10/785,414

(22) Filed: Feb. 25, 2004

Related U.S. Application Data
(63) Continuation of application No. 09/635,468, filed on Aug. 11, 2000.

(30) Foreign Application Priority Data
Aug. 13, 1999 (DE) 199 37 656.5

(51) Int. Cl. A61K 38/48

(52) U.S. Cl. 514/12

(57) ABSTRACT

The use of antithrombin III for the prophylaxis and therapy of oncological diseases and diseases accompanying neovascularization, immune complex-mediated and autoimmune diseases, viral infections, fibrotic and granulomatous diseases, allergic diseases, degenerative diseases of the nervous system and arteriosclerosis and also acute inflammatory diseases and trauma is described.

The invention relates to the use of antithrombin III for the prophylaxis and therapy of diseases.

It is known that antithrombin III (AT) is one of the most important inhibitors of plasma. As a serine protease inhibitor, AT III regulates reactions of the clotting system. Owing to its property of binding heparin and substances related to this, it mediates both the anticoagulant action of such anticoagulants and a protective effect of the endothelium and the matrix lying under it, which carry glycosaminoglycans which are structurally similar to heparin, such as heparan sulfate. The activation of the AT III by these cofactors leads to an acceleration of the reaction with activated proteases, especially with those of the clotting cascade.

The property of binding to heparin is often also utilized to enrich AT III, for example from plasma, and to prepare appropriate concentrates for prophylactic and therapeutic use. Patients having inherited or acquired AT III deficiency states are particularly treated with appropriate products. A significant lack of AT III, particularly during diseases such as sepsis, which is often accompanied by disseminated, intravasal clotting (DIC), associated with a multiorgan failure and shock, often leads to death. In addition to the hemostasis-regulating function of AT III, properties have also already been described which suggest an anti-inflammatory action (1). This hypothesis is based on the finding of the release, mediated by AT III, of prostacyclin from endothelial cells, which inhibits, inter alia, the aggregation of blood platelets.

Moreover, it was shown in in vitro experiments that AT III can reduce the release, stimulated by bacterial lipopolysaccharides, of the cytokine interleukin-6, which is regarded as proinflammatory, and the expression of tissue thromboplastin (tissue factor) from monocytes and endothelial cells.

In animal models, the administration of AT III showed significant, prophylactic and therapeutic effects on mortality in the case of induced sepsis and DIC. Moreover, the reperfusion damage after artificially added ischemia as a result of ligation of organ vessels was decreased.

The effects mediated by AT III can be explained to date by its interaction with its target proteases, soluble and cell- or matrix-bound glycosaminoglycans being involved.

In many diseases, cell-mediated processes, particularly with involvement of leukocytes, play a crucial role. A physiologically significant extent of activated leukocytes contributes to the control of, for example, infection foci. If this reaction, however, is dysregulated, tissue damage occurs, which in the end can lead to organ failure and to death.

The migration of the leukocytes is promoted by the secretion of so-called chemokines, which attract leukocytes along an appropriate concentration gradient to the target area, e.g. to a tissue lesion. Having arrived there, the cells are in an activated state and secrete mediators which can induce or increase further tissue-damaging reactions. Chemokines, such as interleukin-8, interact here with cell membrane receptors on leukocytes and other cells and induce intracellular signal reactions which cause the cell to mobilize and synthesise or secrete proinflammatory substances.

Surprisingly, it has now been found that AT III can reduce or at least control the migration of leukocytes i.e. granulocytes, eosinophils, basophils and lymphocytes/monocytes in the case of stimulation or attraction by a chemokine such as interleukin-8. The leukocytes are influenced here in a manner dependent on the AT III concentration to not react or to react in a weakened manner to a chemokine, which corresponds to a so-called heterologous deactivation. From this, application possibilities emerge for the use of AT III in a number of diseases associated with inflammatory reactions.

The invention therefore relates to the use of AT III for the prophylaxis and therapy of diseases, in particular of oncological diseases and diseases accompanying neovascularization, immune complex-mediated and autoimmune diseases, viral infections, fibrotic and granulomatous diseases, allergic diseases, degenerative diseases of the nervous system and arteriosclerosis and also acute inflammatory diseases and trauma.

Among the immune complex-mediated diseases which can be treated according to the invention, vasculitis and granulomatous diseases are particularly to be emphasized. In the case of the autoimmune diseases, good treatment successes are observed in the case of systemic lupus erythematosus (SLE), rheumatoid arthritis (RA) and pemphigus. Viral infections, in particular HIV too, can also be treated according to the invention by the use of AT III. Among the allergic diseases which can be treated with AT III, bronchial asthma, rhinitis, conjunctivitis and dermatitis can be mentioned especially. Even in the case of degenerative diseases of the nervous system, e.g. in Alzheimer's disease, treatment successes are observed with AT III.

The biochemical and cellular investigations carried out to date with AT III suggest the conclusion that AT III interacts with one or more cellular receptors and leads to a blockade of the chemokine receptors, particularly the so-called CXC receptors, which can then no longer react to chemokines such as interleukin-8. As a result, the chemotactic migration of the leukocytes to these stimuli is absent. From this, a significant in vivo effect can be derived in the sense of a reduction in the intensity of certain illnesses.

The results available until now were obtained experimentally in ex vivo experiments. By incubation of granulocytes with AT III and subsequent attraction by interleukin-8, such as are carried out in vitro in so-called Boyden chambers, a significant reduction in the leukocyte migration was seen, from which prophylactic and therapeutic effects familiar to the person skilled in the art result.

It is indeed known that AT III, in addition to the inherited and acquired deficiency states mentioned, can also already be employed for prophylaxis and therapy in the case of sepsis and DIC (1). Animal-experimental data moreover suggest the use for the reduction of the reperfusion damage after ischemia (2) or in organ transplants, whose rejection reactions were markedly decreased (3).

It was then shown according to the invention that the therapeutic and prophylactic application possibilities of AT III are much more extensive, these results being obtained both with antithrombin III purified from plasma and with recombinantly or transgenically obtained antithrombin III or its mutants or peptides derived therefrom.

The diseases which can be treated prophylactically or therapeutically with AT III can be divided into

1. acute cell-mediated inflammatory reactions and

2. chronic cell-mediated inflammatory reactions.

In particular, these are to be understood as meaning:

urticaria and angioedema

asthma

pulmonary emphysema

pemphigus

vasculitis

graft-versus-host disease

granulomatous inflammatory diseases

rheumatoid arthritis

systemic lupus erythematosus

gout

neutrophilic dermatoses

fibrotic liver diseases

inflammatory neoplasias

diseases associated with neovascularization

viral infections, whose mechanism of penetration into the target cells takes place by means of chemokine receptors, such as HIV

neurodegenerative illnesses

vascular inflammations, e.g. atherosclerosis, which is accompanied by a chemokine-mediated infiltration by leukocytes.

Depending on the particular inflammatory reaction, AT III can be employed intravenously, subcutaneously, intradermally/intramuscularly or topically.

The invention is explained in greater detail by the following example.

### EXAMPLE

Neutrophilic granulocytes were prepared from whole blood of healthy donors. For this, methods familiar to the person skilled in the art, such as separation by centrifugation in suitable media such as Ficoll, were used. After the subsequent lysis of remaining erythrocytes, the granulocytes were isolated by centrifugation again. After determination of the cell counts, the granulocytes were investigated without delay in the chemotaxis test.

This test was carried out in so-called Boyden chambers. In principle, the method is based on attracting the cells through a membrane filter in a two-compartment system by construction of a chemotactic gradient. Customarily, the cells are in this case pipetted into the “upper” chamber, the chemokine into the “lower” chamber. Along the gradient, the cells penetrate into the pores of the filter. With a sufficiently long incubation, the cells migrate through the filter and are counted on its bottom or in the lower chamber. In the experiment described, however, the cell count of the cells which had migrated into the filter was determined microscopically after fixing and staining. The chemotactic index in this case represents the effect of the chemoattractant which results from the quotient of the cell counts of the migration induced by the chemokine and the undirected migration (chemokinesis).

For the investigation of the cell migration-modulating effect, the granulocytes were incubated at 37° C. for 15 min with increasing concentrations of AT. After washing the cells a number of times, these were employed in the chemotaxis test. The chemokines used were IL-8 (1 nM) or C5a (0.1 nM). After incubation at 37° C. for 30 min, the filter inserts were washed, and the cells were fixed, stained and counted. The chemokinesis (without attractant) and the chemotaxis with AT III-untreated cells (positive control) were also determined. The chemotactic index was determined as described above.

Each experimental series was carried out in 4 parallel batches, cells from in each case 3 healthy donors being used.

The result is illustrated in the following table:

TABLE
Dose-dependent deactivation of the chemotaxis of neutrophilic granulocytes to
interleukin-8 (1 nM), Formyl-Met-Leu-Phe (10 nM) or complement 5a (0.1 nM)
by AT III/Kybernin ®

| Priming | | | Chemotaxis index | | | |
|---|---|---|---|---|---|---|
| 15 Min. | Interleukin-8 | | Formyl-Met-Leu-Phe | | Complement 5a | |
| AT III/ | (1 nM) | | (10 nM) | | (0.1 nM) | |
| Kybernin | | Standard | | Standard | | Standard |
| (log U) | Mean value | error | Mean value | error | Mean value | error |
| Control | 1.823 | 0.194 | 1.625 | 0.036 | 2.136 | 0.021 |
| −6 | 1.494 | 0.123 | 1.335 | 0.069 | 2.214 | 0.008 |
| −5 | 1.490 | 0.009 | 1.383 | 0.043 | 2.018 | 0.057 |
| −4 | 1.513 | 0.086 | 1.368 | 0.067 | 2.253 | 0.086 |
| −3 | 1.395 | 0.058 | 1.327 | 0.052 | 2.198 | 0.184 |
| −2 | 1.334 | 0.078 | 1.228 | 0.042 | 2.023 | 0.152 |
| −1 | 1.225 | 0.034 | 1.151 | 0.061 | 1.926 | 0.123 |
| 0 | 1.160 | 0.043 | 1.118 | 0.049 | 1.373 | 0.118 |
| 0.5 | 1.083 | 0.021 | 1.068 | 0.038 | 1.315 | 0.109 |
| 1 | 1.021 | 0.019 | 1.051 | 0.030 | 1.302 | 0.088 |

Mean values and standard error of n = 3;
Medium served as a control;
Kybernin vs. IL-8, p < 0.0001, vs. Formyl-Met-Leu-Phe p < 0.001, vs. C5a p < 0.005; Kruskal-Wallis test.

### BIBLIOGRAPHY

1. K. Okajima, Biomed. Progr. 9: 42-44 (1996).

2. Ostrovski, L.; Woodman, R. C.; Payne, D.; Teoh, D.; Ischemia/Reperfusion and Antithrombin III, Circulation Vol. 96, No. 7, 2302-2310 (1997).

3. Zuo, X. J., Okada, Y., Nicolaidou, E.; Toyoda, T.; Marchevsky, A.; Matloff, J. M. and Jordan, S. C.; Transplantation Proceedings, 31, 847-848 (1999).

1-8. (Canceled)

9. A method of reducing chemokine-mediated migration of leukocytes in an individual suffering from an inflammatory disease or disorder, comprising administering antithrombin III to said individual in an amount sufficient to interact with one or more cellular receptors to effect a blockade of chemokine receptors, and reduce chemokine-mediated migration of leukocytes,
wherein said inflammatory disease or disorder is
a) an oncological disease, a disease accompanying neovascularization, a viral infection, a fibrotic disease or disorder, vasculitis, a granulomatous disease, a degenerative disease of the nervous system, atherosclerosis, gout; or
b) an autoimmune disease, provided that the autoimmune disease in not arthritis, or
c) an allergic disease, provided that the allergic disease or disorder is not asthma.

10. The method of claim 9, wherein the disease or disorder is vasculitis or a granulomatous disease.

11. The method of claim 9, wherein the disease or disorder is an autoimmune disease or disorder.

12. The method of claim 11, wherein the autoimmune disease is systemic lupus erythematosus or pemphigus.

13. The method of claim 9, wherein the disease or disorder is a viral infection.

14. The method of claim 13, wherein the viral infection is caused by a human immunodeficiency virus.

15. The method of claim 13, wherein the viral infection involves viral penetration into target cells via chemokine receptors.

16. The method of claim 9, wherein the disease or disorder is an allergy or allergic reaction.

17. The method of claim 16, wherein the allergy or allergic reaction is urticaria or angioedema.

18. The method of claim 16, wherein the allergy or allergic reaction is rhinitis, conjunctivitis, or dermatitis.

19. The method of claim 9, wherein the disease or disorder is a degenerative disease of the nervous system.

20. The method of claim 19, wherein the degenerative disease of the nervous system is Alzheimer's disease.

21. The method of claim 9, wherein the disease or disorder is an oncological disease or disorder.

22. The method of claim 21, wherein the oncological disease or disorder is an inflammatory neoplasia.

23. The method of claim 9, wherein the disease or disorder is a disease or disorder accompanying neovascularization.

24. The method of claim 9, wherein the disease or disorder is atherosclerosis accompanied by a chemokine-mediated infiltration by leukocytes.

25. The method of claim 9, wherein the disease or disorder is gout.

26. The method of claim 9, wherein the disease or disorder is a fibrotic disease or disorder.

27. The method of claim 9, comprising administering antithrombin III in an amount sufficient to reduce interleukin-8-mediated migration of leukocytes.

28. The method of claim 9, wherein said antithrombin III is obtained from plasma or a recombinant or transgenic source.

29. The method of claim 9, wherein said antithrombin III is administered intravenously, subcutaneously, intradermally, intramuscularly, or topically.
